(19) United States
(12) Patent Application Publication
(10) Pub. No.: US 2007/0074929 A1
(43) Pub. Date: Apr. 5, 2007

(54) Disposable stethoscope cover construction

(76) Inventors: Loretta Annette Garcia, Austin, TX (US); Inge Anna Williams, Austin, TX (US)

Correspondence Address: INGE A. WILLIAMS, 2500 BLUFF VIEW DRIVE, AUSTIN, TX, 78704, US

(21) Appl. No.: 11/238,904

(22) Filed: Sep. 30, 2005

(51) Int. Cl. A61B 7/02

(52) U.S. Cl. 181/131

(57) ABSTRACT

A disposable stethoscope cover construction for covering the diaphragm, bell, and neck portions of a conventional stethoscope wherein the cover construction includes a contoured sheet of material made from a thin, resilient, fluid-impermeable fabric and including: an enlarged diameter generally circular portion dimensioned to surround and cover the diaphragm portion of a stethoscope; a reduced diameter generally circular portion dimensioned to surround and cover the bell portion of the stethoscope; and, a relatively narrow neck portion dimensioned to partially surround the neck portion of the stethoscope wherein the periphery of the contoured sheet of material is elasticized to permit the frictional engagement of the cover construction with the stethoscope. Combined with the disposable nature and ease of application and removal, this particular stethoscope cover protects the diaphragm as well as the bell and neck portions of the stethoscope, thereby preventing direct contact between stethoscope and patient, and consequently minimizing the spread of infection from patient to patient.

### CROSS REFERENCE TO RELATED APPLICATIONS

Not applicable

### STATEMENT REGARDING FEDERALLY SPONSORED RESEARCH OR DEVELOPMENT

Not applicable

### REFERENCE TO MICROFICHE APPENDIX

Not applicable

### TECHNICAL FIELD AND BACKGROUND OF THE INVENTION

The present invention relates to the field of stethoscope covers in general and in particular to a disposable stethoscope cover that surrounds the bell, neck and diaphragm portions of the stethoscope. It is fabricated from a thin, fluid impermeable material, which functions as a barrier between stethoscope and patient, without interfering with the auscultatory properties of the stethoscope. Combined with a simple and practical design, it can be easily applied and removed from the stethoscope in one swift motion.

Over two million hospital-acquired infections occur annually in the U.S. These “nosocomial infections” result in substantial morbidity, mortality, and cost. In numerous studies, stethoscopes have been shown to be a major carrier of potentially hazardous microorganisms. In one particular study, two hundred stethoscopes from four hospitals were tested for contamination. Eighty percent were contaminated with microorganisms. Seventeen distinct species of microorganisms were isolated from stethoscopes, and more than half of the staphylococci isolated were resistant to methicillin (Known as “MRSA”, Methicillin Resistant Staph Aureus). (Smith M A et al, Contaminated Stethoscopes, Arch Intern Med. 1996, 156:82-4).

A similar study demonstrated that enterococcal species can survive on stethoscope surfaces for 30 minutes and that VRE (Vancomycin Resistant Enterococcus) can survive on environmental surfaces >18 hours. (Weber, David, MD et al, Role of Environmental Contamination in the Transmission of VRE, Infection Control and Hospital Epidemiology, May 1997, Vol. 18, 5.)

Stethoscopes are standard equipment in health care and they are rarely disinfected properly. For LOW-LEVEL disinfection with phenolics, ammonium compounds, and alcohol, exposure times of three to ten minutes are needed (Weber, David MD, 1997). However, this is unrealistic and not very practical.

Moreover, in a research study by Jan Nick, RNC, PhD (Organic Buildup and Residual Blood on Infant Stethoscopes in Maternal-Infant Areas, JOGNN, March 1999, 1-10) the presence of residual blood and organic matter on “clean” stethoscopes was identified. Of 97 clean infant stethoscopes, 80% of labor and delivery and 72% of nursery stethoscopes had organic buildup on the diaphragm. Seventy-six percent of L&D stethoscopes were positive for blood, as compared to 46% of nursery stethoscopes. The study concludes that traditional or routine cleaning procedures may prove ineffective in removing blood and other body fluids, providing a media for microorganism proliferation.

In a study conducted in 1997, M H Kollef evaluated the relationship between nosocomial infection and clinical outcomes following cardiac surgery. Six hundred consecutive patients undergoing cardiac surgery were studied. One hundred thirty-one (21.7%) patients acquired at least one nosocomial infection following cardiac surgery (The Impact of Nosocomial Infections on Patient Outcomes Following Cardiac Surgery, Kollef M H et al, Chest, 1997 September, 112 (3):666-75). Placement of a contaminated stethoscope on the chest of a fresh post-cardiac patient likely contributes to propagation of nosocomial infections in this patient population.

Of significance, stethoscopes play a unique role in health care delivery in that most often, the same stethoscope is used by a health care provider (physician, nurse, respiratory therapist, etc.) to examine multiple patients, not only in different units or floors of a hospital, but in many instances, multiple hospitals as well. The spread of infection via contaminated stethoscopes poses an enormous problem.

To help overcome this problem, several research articles have suggested the implementation of barriers or DISPOSABLE STETHOSCOPE COVERS. The elimination of the stethoscope as a source of disease propagation could substantially improve patient outcomes and decrease mortality, morbidity, and medical care costs.

### DESCRIPTION OF RELATED ART

As can be seen by reference to the following U.S. Patent Nos. 5,747,751; 5,949,032; 5,808,244; and, 5,587,561, the prior art is replete with myriad and diverse protective devices for stethoscopes.

While all of the aforementioned prior art constructions are adequate for the basic purpose and function for which they have been specifically designed, they are uniformly deficient with respect to their failure to provide a simple, efficient, and inexpensive disposable protective cover construction for stethoscopes that can be installed on and removed from a stethoscope with one hand, thereby virtually insuring its use in its intended manner by time-constrained health care workers. In addition, this particular stethoscope cover construction covers the diaphragm as well as the bell and neck portions of the conventional stethoscope, thereby preventing direct contact between the stethoscope and the patient.

As many health care professionals are aware, the repeated use of stethoscopes on a large number of different patients under different circumstances and suffering from different diseases and illnesses is believed to be a major contributing factor in the transmission of pathogens among patients. Contamination occurs not just between various hospital rooms, but also between various buildings, such as doctor's offices, clinics, different hospitals with different locations. This is especially worrisome with the drug-resistant pathogens developing in ever-increasing numbers and severity.

As a consequence of the foregoing situation, there has existed a longstanding need in the medical profession for a new and improved protective cover construction for stethoscopes which sacrifices full stethoscope coverage in exchange for ease in installation and disposal which should insure its widespread use and acceptance (just like protective gloves have become the necessary norm and standard before touching any patient). The provision of a cover construction is the stated objective of the present invention.

### BRIEF SUMMARY OF THE INVENTION

As pointed out in the previous section, the spread of infection via contaminated stethoscopes poses an enormous problem. The object of our invention is to help overcome this problem by introducing a disposable cover construction for stethoscopes, one that is made from a fabric presently being used for its pathogen-resistant properties in the manufacture of isolation gowns and operating-room shoe covers in today's health care settings. This particular material is resilient, liquid impermeable, inexpensive, and causes minimal interference with sound transmission.

Furthermore, our invention has a practical and simple design, which is inexpensive to produce and can be inexpensively mass-packaged (similar to that of disposable gloves, such as boxes of 100's).

Another advantage of our invention is the added bonus of comfort. The stethoscope cover prevents the discomfort caused by placement of a cold stethoscope against bare skin. This is especially beneficial when assessing children or newborn babies.

Moreover, while being latex-free and non-allergenic, our stethoscope cover construction is also non-sticking, therefore leaving no adhesive residue on the head of the stethoscope.

The disposable cover construction is comprised of a contoured sheet of the stated material with an external periphery that is elasticized to create two different-sized, generally cup-shaped portions that are adapted to resiliently engage in a tensioned condition over the skin-contacting surfaces of the stethoscope. The diaphragm as well as the bell and neck portions are protected, thereby preventing direct contact between the stethoscope and the patient, consequently minimizing the transmission of microorganisms from one patient to another.

The virtue of this protective covering, combined with the disposable nature and ease of application and removal, should go a long way towards minimizing the transfer of pathogens in any medical environment.

### BRIEF DESCRIPTION OF THE SEVERAL VIEWS OF THE DRAWINGS

These and other attributes of the invention will become clear upon a thorough study of the following description of the best mode for carrying out the invention, particularly when reviewed in conjunction with the drawings, wherein:

FIG. 1 is a perspective view of a conventional stethoscope showing the protective cover outlined in phantom;

FIG. 2 is a top plan view of the contoured sheet of material prior to the elasticization of the peripheral edges of the sheet of material;

FIG. 3 is a perspective view of the finished version of the stethoscope cover construction;

FIG. 4 is a top plan view of the stethoscope cover partially engaged with the diaphragm portion of the stethoscope;

FIG. 5 is a perspective view of the arrangement depicted in FIG. 4, illustrating the application of the cover construction over the bell portion of the stethoscope;

FIG. 6 is a side plan view of the stethoscope cover construction fully engaged with the bell, neck and diaphragm portions of the stethoscope.

### DETAILED DESCRIPTION OF THE INVENTION

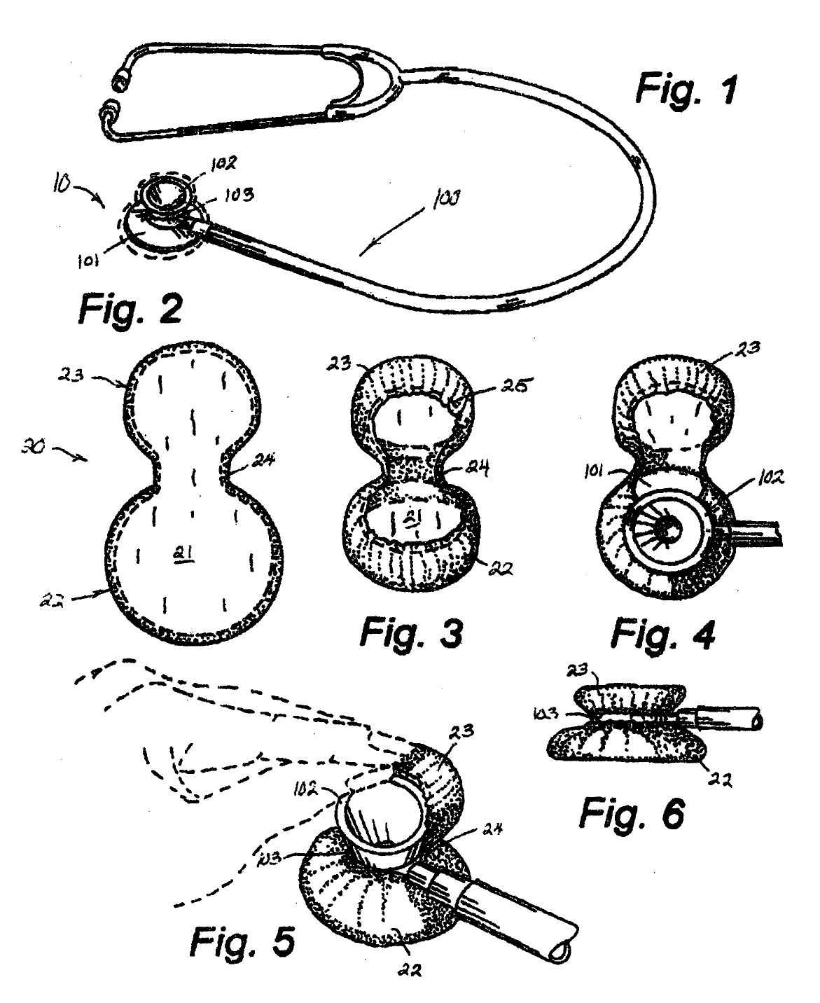

As can be seen by reference of the drawings, and in particular to FIG. 1, the disposable stethoscope cover construction that forms the basis of the present invention is designated generally by the reference number 10 and is specifically designed for use with the conventional stethoscope 100 having a diaphragm 101 and a bell 102 connected together by a narrow neck 103.

As shown in FIGS. 2 and 3, the protective cover construction 10 comprises a contoured sheet of material 20 made from a thin, water-resistant, spun polypropylene material 21 with germ-resistant properties (presently being used in hospitals for protective coverings such as isolation gowns), wherein the minimal thickness of the material 20 will not degrade the acoustical qualities of the stethoscope 100.

In addition, the contoured sheet of material 20 is provided with: an enlarged diameter generally circular portion 22 dimensioned to extend beyond and surround the diaphragm 101 of the stethoscope 100; a reduced diameter generally circular portion 23 dimensioned to extend beyond and surround the bell 102 of the stethoscope 100; and, a relatively narrow neck portion 24 dimensioned to partially surround the neck portion 103 of the stethoscope 100.

Still referring to FIGS. 2 and 3, it can be seen that the generally two dimensional characteristics of the contoured sheet of material 20 are dramatically altered when the periphery of the sheet of material 20 is stitched with an elastic thread 25 which produces two different-sized, cup-shaped portions 22 and 23, which remain connected by a curved neck portion 24.

In the preferred method of use, a health care professional would insert one side of the stethoscope diaphragm into the free end of the larger of the two cup-shaped portions 22 and then by grasping the free end of the smaller of the cup-shaped portions 23 loop the smaller of the cup-shaped portions 23 over the stethoscope bell 102 thereby providing a protective cover to the bell, diaphragm and neck portions of the stethoscope.

In this manner, each time a physician or nurse wants to employ the stethoscope 100 on a different patient, it is very simple and easy to remove and discard one protective cover 10 from the stethoscope 100 and replace it with a fresh new stethoscope cover 10 thereby substantially reducing the possibility that contaminated fluids and/or resistant germs can be transferred from one patient to the next.

Although only an exemplary embodiment of the invention has been described in detail above, those skilled in the art will readily appreciate that many modifications are possible without materially departing from the novel teachings and advantages of this invention. Accordingly, all such modifications are intended to be included within the scope of this invention as defined in the following claims.

Having thereby described the subject matter of the present invention, it should be apparent that substitutions, modifications and variations of the invention are possible in light of the above teachings. It is therefore to be understood that the invention as taught and described herein is only to be limited to the extent of the breadth and scope of the appended claims.

What we claim is:

1. A method for preventing transmission of microorganisms by controlling the contamination of stethoscopes, a health care professional's most common tool, said method comprising the steps of:
1. A disposable stethoscope cover construction for covering the diaphragm, bell, and neck portions of a conventional stethoscope wherein the cover construction comprises a cover unit including a contoured sheet of material having an enlarged diameter generally circular portion, a reduced diameter generally circular portion and a relatively narrow neck portion which is disposed intermediate said enlarged and reduced diameter circular portions.
2. The cover construction as in claim 1; wherein, said enlarged diameter circular portion has a diameter substantially greater than the diameter of the diaphragm portion of the conventional stethoscope.
3. The cover construction as in claim 2; wherein, said reduced diameter circular portion has a diameter substantially greater than the diameter of the bell portion of the conventional stethoscope.
4. The cover construction as in claim 3; wherein, said contoured sheet of material has an external periphery that is elasticized to produce a bunching effect around said periphery.
5. The cover construction as in claim 4; wherein, the external periphery of said contoured sheet of material is sewn with an elastic thread to produce the bunching effect.
6. The cover construction as in claim 5; wherein, said contoured sheet of material consists of a thin, liquid impermeable material, currently used in health care settings to provide a barrier between a patient and a health care worker to prevent transmission of microorganisms (such as isolation gowns or sterile shoe covers for the O.R.).
7. A method of making a disposable stethoscope cover construction for covering the diaphragm, bell, and neck portions of a conventional stethoscope comprising the steps of
a) severing a sheet of the liquid impermeable material so that the severed sheet of material has a contour which includes an enlarged diameter generally circular portion, a reduced diameter generally circular portion and a relatively narrow neck portion disposed intermediate the enlarged and reduced diameter generally circular portions; and,
b) stitching the periphery of the severed portion of the sheet of material with a straight stitch on a sewing machine, using cotton thread on the spool and an elastic thread on the bobbin. This technique gathers the edges and creates a bunching effect around the periphery of the severed portion of the sheet of material. This elasticized edge is placed in a tensioned condition over and covering the patient-contacting portion of the stethoscope, thereby preventing fluids and other extraneous matter that may be present on the skin of a patient from contacting said portion of the stethoscope. Consequently, this disposable protective covering of the stethoscope reduces the risk of contamination from patient to patient.
